(19) United States
(12) Patent Application Publication
(10) Pub. No.: US 2005/0048546 A1
(43) Pub. Date: Mar. 3, 2005

(54) Multiplexed molecular beacon assay for detection of human pathogens

(76) Inventors: Sharron Penn, San Carlos, CA (US); Michael Sha, Castro Valley, CA (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/889,466

(22) Filed: Jul. 12, 2004

Related U.S. Application Data
(60) Provisional application No. 60/486,477, filed on Jul. 11, 2003.

(51) Int. Cl. C12Q 1/68; C12M 1/34

(52) U.S. Cl. 435/6; 435/287.2

(57) ABSTRACT

Encoded metal nanoparticles conjugated to oligonucleotides, and methods for their use are described.

### CROSS REFERENCE TO RELATED APPLICATIONS

This application claims the benefit of U.S. Provisional Patent Application Ser. No. 60/486,477, filed Jul. 11, 2004, entitled “Highly Multiplexed Nanoparticle-Based Assays,” which is incorporated by reference herein in its entirety.

### FIELD OF THE INVENTION

The field of this invention is molecular biology, particularly nucleic acid hybridization, and protocols for the identification of target nucleic acids.

### BACKGROUND OF THE INVENTION

The use of fluorescence quenching as a detection method in biological assays is widespread and includes the use of molecular beacons, a technology first described in 1996. Tyagi, S. and Kramer, F. R., “Molecular Beacons: probes that fluoresce upon hybridization” Nature Biotechnol. 1996, 14, 303-308. Molecular beacons typically use a fluorophore reporter dye and a non-fluorescent quencher chromophore. While in close proximity, the fluorophore is quenched by the energy transfer to the non-fluorescent chromophore. However, separating the fluorophore and the quencher results in a fluorescent signal. Molecular beacons have been used in a variety of assay formats, including the monitoring of nucleus activity, the detection of pathogens and SNP detection .

An assay using a fluorescent energy transfer system, such as molecular beacons, does not require the target nucleic acid to be labeled, nor does the target nucleic acid have to be separated from the other components of the assay. For example, fluorescence quenching has been used to monitor the amplification of the target sequences in RT-PCR on a cycle-by-cycle basis.

Quenching in molecular beacons is commonly achieved with the nonfluorescent chromophore, 4-(4′-dimethylaminophenylazo) benzoic acid (DABCYL). Under some circumstances, organic fluorophores are quenched when in very close proximity to metallic surfaces. Lakowicz, J. R., “Radiative Decay Engineering: Biophysical and Biomedical Applications” Anal. Biochem. 2001, 298, 1-24. The presence of metals provides alternative non-radiative energy decay paths that can change the fluorescence quantum yield of a fluorophore. At close distances (<50 angstroms), fluorescence is quenched while at intermediate distances (75 to 100 angstroms), it is enhanced. The phenomena is well documented for Ag and Au films quenching the fluorescence of Rhodamine dyes.

A fluorophore will function and quench appropriately, when linked to an Au surface. See Du, H., Disney, M., Miller, B., and Krauss, T., “Hybridization-Based Unquenching of DNA Hairpins on Au Surfaces: Prototypical “Molecular Beacon” Biosensors” J. Am. Chem. Soc. 2003,125, 4012-4013. Quenched fluorophore assays on Au colloids can distinguish oligonucleotides with single base mismatches. See Maxwell, D. J., Taylor, J. R., and Nie, S., “Self-assembled nanoparticle probes for recognition and detection of biomolecules” J. Am. Chem. Soc. 2002, 124, 9606-9612; Dubretret, B., Calame, M., and Libchaber, A. J., “Single-mismatch detection using gold-quenched fluorescent oligonucleotides” Nature Biotechnol. 2001, 19, 365-370. This work is possible because fluorescent dyes will reversibly absorb onto colloidal Ag and Au. Nie, S. and Emory, S. R., “Probing Single Molecules and Single Nanoparticles by Surface-Enhanced Raman Scattering” Science 1997, 275, 1102-1106; Krug, J. T., II, Wang, G. D., Emory, S. R., and Nie, S., “Efficient Raman Enhancement and Intermittent Light Emission Observed in Single Gold Nanocrystals” J. Am. Chem. Soc. 1999, 121, 9208-9214. When oligonucleotides are single stranded, they have flexibility and can form looped structures due to their attraction to the Au surface. However, when hybridized, the now double stranded oligonucleotides are rigid such that the fluorescent dye cannot interact with the surface.

There are a number of assays available to interrogate DNA and determine the sequence of bases. These assays range from de novo DNA sequencing of many hundreds of bases at a time to the interrogation of a single base, as in the case of SNP detection. In the majority of these assays, labels are needed to identify a particular product or event from among the thousands of molecules and events also present in the cell or biological extract under interrogation. While there are a few analytical techniques that can directly detect the native molecule, such as mass spectrometry and nuclear magnetic resonance spectroscopy , these often require very specific sample preparation, highly sophisticated and expensive equipment, and often do not work in complex biochemical backgrounds. Therefore, in complex biological systems, the molecule of interest is typically labeled in some way to make it “visible” in order to be assayed. Common labels used in biology include radioactivity, organic fluorophores and quantum dots. However, labeling the molecule being interrogated adds a level of complexity to an assay, thereby making it more difficult to perform properly and consistently, more difficult to turn into a “kit” or product, and more difficult to make the assay field portable and robust due to the additional steps involved. Thus, it would be desirable to have an assay that did not involve a labeling step.

Multiplexing affords the ability to make two or more measurements simultaneously. This has a number of advantages. It reduces the time and cost to collect the measurement. It can often reduce the amount of sample needed to acquire the measurement. More importantly, it allows data to be reliably compared across multiple experiments. Additionally, multiplexing can add confidence to the measurement results through the incorporation of multiple internal controls. Thus, it would be desirable to have an assay that was capable of being used for multiplexed analysis.

Many currently used assays require specialized equipment, such as the massively parallel capillary electrophoresis devices used in DNA sequencing to specialized readers for RT-PCR. However, in many instances, an instrument dedicated to a single experiment may not be feasible for reasons of cost, resources and/or space. For example, the Mobile Analytical Laboratories (MAL) of the First Responder Units are called to the scenes of potential bioterrorism incidents. The MAL may include a GC-MS, dip-test reagents to detect blister agents, a dosimeter to detect radioactivity, air samplers, a PCR thermocycler with reagents to detect pathogens including anthrax and plague via real time RT-PCR, and a fluorescence microscope that is interfaced to a camera with wireless connection to e-mail images to agencies such as the CDC. An additional assay cannot be performed by such a Unit if it requires an additional specialized piece of equipment.

### SUMMARY OF THE PRESENT INVENTION

The present invention provides a particle-based multiplexable diagnostic assay for DNA detection using fluorescence quenching by metallic surfaces. The present invention also provides a multiplexed diagnostic assay for detection of polynucleotides and oligonucleotides (e.g., DNA, RNA).

### BRIEF DESCRIPTION OF THE FIGURES

FIG. 1 is a schematic depiction of a multiplexed assay embodiment of the present invention the detection of a pathogenic polynucleotide.

FIG. 2 is a bar chart plotting median fluorescence intensity of various Nanobarcodes® particles that have been conjugated to carboxytetramethylrhodamine (TAMRA) labeled oligonucleotides. In FIG. 3A, the particles are conjugated to a TAMRA labeled 32-mer oligonucleotide (M1) and the fluorescence is shown for the conjugated particles in the presence of complementary M1C (grey bar), noncomplementary M2C (black bar) and water (white bar). In FIG. 3B, the particles are conjugated with a different TAMRA labeled oligonucleotide (M2) and the fluorescence is shown for the conjugated particles in the presence of noncomplementary M1C (grey bar), complementary M2C (black bar) and water (white bar).

FIG. 3 schematically illustrates possible orientations of thiol-DNA-fluorophore species on Nanobarcodes particles.

FIG. 4 schematically illustrates an alternative assay design involving two different fluorophores

FIG. 5 schematically illustrates possible orientations of Nanobarcodes particle conjugated, thiol-DNA species (flourophore labeled and unlabeled) hybridized to target nucleic acid (flourophore labeled and unlabeled).

FIG. 6 schematically illustrates the binding of a flourophore labeled target nucleic acid to the thiol-DNA-fluorophore species conjugated to a Nanobarcodes particle.

FIG. 7 is a bar chart plotting median fluorescence intensity of various Nanobarcodes® particles that have been conjugated either TAMRA-labeled HIV probe oligonucleotide or TAMRA-labeled HCV oligonucleotide.

FIG. 8 is a bar chart plotting median fluorescence intensity of various Nanobarcodes® particles that have been conjugated to an HIV probe sequence (HIV mb2), an HCV probe sequence (HCV mb2) or an HBV probe sequence (HBV mb2).

### DETAILED DESCRIPTION OF INVENTION

The present invention provides a simple assay that can be performed on non-specialized equipment. The assay may be run in a multiplexed format. The assay has utility with respect to a number of fields, including pathogen monitoring, environmental monitoring, healthcare diagnostics, and in field food-borne pathogen detection. The present invention allows “label-free,” multiplexable DNA analysis assays and does not require a dedicated and specialized instrument for analysis. The present invention enables a larger number of analyses to be performed faster, in non-laboratory based environments and by non-technical operators. In addition, the assay has high specificity and sensitivity.

It is to be noted that the term “a” or “an” entity refers to one or more of that entity; for example, a protein refers to one or more proteins or at least one protein. As such, the terms “a” (or “an”), “one or more” and “at least one” can be used interchangeably herein. It is also to be noted that the terms “comprising”, “including”, and “having” can be used interchangeably. As used herein, the term “oligonucleotides” refers to a short polymer composed of deoxyribonucleotides, ribonucleotides or any combination thereof. These oligonucleotides are at least 5 nucleotides in length, but may be about 20 to about 100 nucleotides long. In certain embodiments, the oligonucleotides are joined together with a detectable label, which includes a fluorophore.

In a typical multiplexed embodiment of the assay of the present invention, each Nanobarcodes particle “flavor” (that is, each particle having a particular encoding) is conjugated with a different oligonucleotide, the oligonucleotide being labeled with a fluorescence dye. A record is kept of which oligonucleotide probes is attached to which uniquely coded particles. Upon addition of DNA (or other target polynucleotide or oligonucleotide) to the assay, there is hybridization with the complementary sequence conjugated to one or more of the nanobarcode flavors. The resulting hybrid is comparatively rigid and causes the fluorophore to move away from the surface, and therefore is no longer quenched. When analyzed using a fluorescence microscope, a Nanobarcodes particle bearing the unquenched fluorophore will apprear bright while the other Nanobarcodes particles will appear dark. Decoding of the flavor of the bright Nanobarcodes particles indicates which DNA sequence was present.

Oligoncleotides used according to this invention comprise at least a single-stranded nucleic acid sequence that is complementary to a desired target polynucleotide or oligonucleotide (either or both of which shall be referred to herein as a “target nucleic acid”), and a detectable label for generating a signal. Some oligonucleotides include complementary nucleic acid sequences, or “arms,” that reversibly interact by hybridizing to one another under the conditions of detection when the target complement sequence is not bound to the target. In some cases, these oligonucleotides are referred to as “hairpin” oligonucleotides. Hairpin oligonucleotides are described elsewhere in this disclosure. When the detectable label is a fluorophore, the oligonucleotide may be (or function in a similar fashion to) a molecular beacon. Molecular beacons are single-stranded oligonucleotide hybridization probes that form a stem-and-loop (hairpin) structure. Molecular beacons typically use a fluorophore reporter dye and a non-fluorescent quencher chromophore. While in close proximity, the fluorophore is quenched by the energy transfer to the non-fluorescent chromophore. However, separating the fluorophore and the quencher results in a fluorescent signal. The oligonucleotides used in the present invention utilize an encoded metallic nanoparticle as the non-fluorescent quencher.

The oligonucleotide used need not be a hairpin oligonucleotide. Because single-stranded DNA has a flexible backbone, the DNA is conformationally flexible. Previous studies have shown the many fluorescent dyes spontaneously adsorb on gold and silver surfaces. In this case then, oligonucleotides may be conjugated to a encoded metal particle on one end, and have a fluorphore in close proximity to the surface of the encoded metal particle on the other end, and where the DNA does not contact the surface of the metal particle, but rathers forms an archlike structure. Both the hairpin (“stem-and-loop”) configuration and non-hairpin (“arched”) configuration are within the scope of the present invention. Although preferred embodiments are described herein with respect to Nanobarcodes particles, the invention is not so limited. The scope of the invention extends to any encoded metal particle or encodable metal particle, such as Nanobarcodes particles composed of metal, or that have one or more metal segments.

The oligonucleotide-conjugated encoded metal particles of the present invention have many applications. They can be used in situations in which ordinary molecular beacons have been used, such as in real-time PCR detection; single-nucleotide mutation screening; allelic discrimination, that is, differentiatiation between homozygotes and heterozygotes; diagnostic clinical assays in which the oligonucleotide-conjugated encoded metal particles, in conjunction with PCR, can be used to detect the presence and abundance of, for example, certain viruses or bacteria in a tissue or blood sample. These methods are well-known to those of ordinary skill in the art.

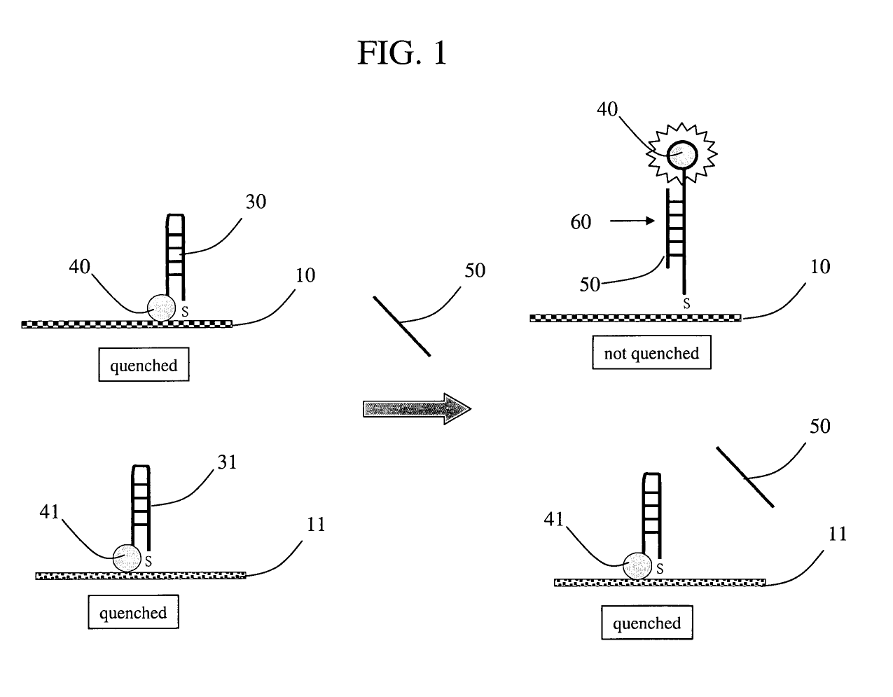

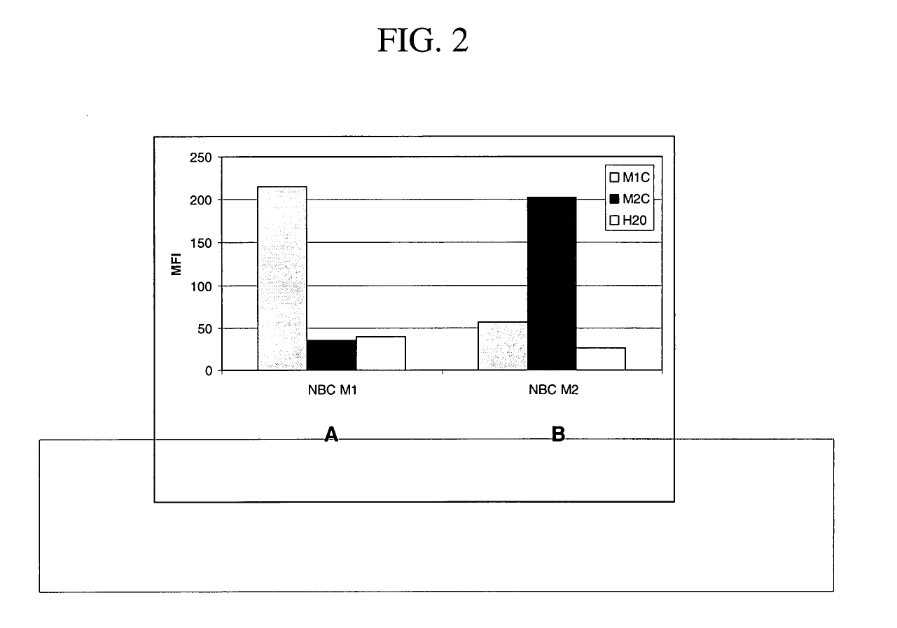

A simple multiplexed assay (two-plex) may be used to differentiate between two different pathogens. Referring to FIG. 1, two Nanobarcodes particles of different striping patterns are employed. The first particle 10 is conjugated to the first probe oligonucleotide 30, complementary to DNA from Pathogen A. The second particle 11 is conjugated to the second probe oligonucleotide 31, complementary to DNA from Pathogen B. The probe oligonucleotides are labeled with a fluorescent dye at a distance from the attachment to the particle. The first probe oligonucleotide is labeled with a first fluorescent dye 40 and the second probe oligonucleotide is labeled with a second fluorescent dye 41. Typically, the first and second fluorescent dyes are the same and detection step is performed at a single wavelength. However, in other embodiments, the first and second fluorescent dyes are different.

Upon addition of DNA 50 from Pathogen A, hybridization between the pathogen DNA and the complementary sequence 30 occurs. The resulting DNA structure 60 is rigid and therefore causes the fluorophore 40 to be moved away from the quenching surface 20 of the first particle 10. Upon analysis with a fluorescence-based microscope, one particle will appear bright due to the unquenched fluorescence while the other particle will appear dark because its fluorescence will be quenched. Using reflectance mode, the striping pattern of the bright particle may be discerned. In this way, the bright particle will be identified as the first particle 10, and accordingly the oligonucleotide that hybridized to the Pathogen will be identified as the first oligonucleotide 20. The very large number of possible Nanobarcode patterns allows for very high multiplexing using only a single fluorescent dye and without the need to label target nucleic acids.

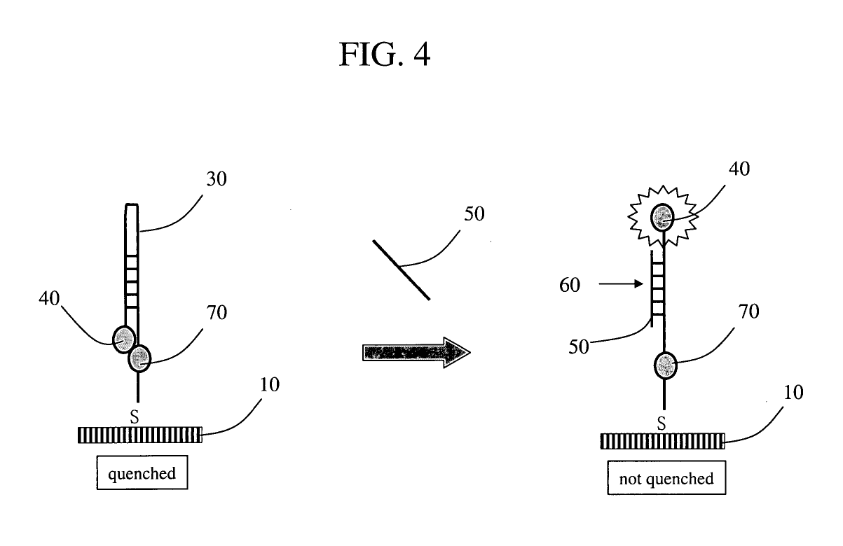

An alternative embodiment as depicted in FIG. 4. As described above, a nanobarcodes particle 10 is conjugated to a probe oligonucleotide 30 that is labeled with a fluorophore 40. Here, however, a quencher molecule 70 has been introduced into the oligonucleotide 30, such that the metallic surface of the Nanobarcodes particle 10 is no longer needed as a quencher, but is used only as an encoded solid support. Upon addition of DNA 50 from Pathogen A, hybridization between the pathogen DNA and the complementary sequence 30 occurs. The resulting DNA structure 60 is rigid and therefore causes the fluorophore 40 to be moved away from the quenching molecule 70 of the particle 10. Upon analysis with a fluorescence-based microscope, the particle will appear bright due to the unquenched fluorescence. Using reflectance mode, the striping pattern of the bright particle may be discerned. In this way, the bright particle will be identified as the first particle 10, and accordingly the oligonucleotide that hybridized to the Pathogen will be identified as the first oligonucleotide 20.

Nanobarcodes® particles are encodeable, machine-readable, durable, sub-micron sized striped metallic rods, fabricated using electroplating methods borrowed from the electrochemical industry. The power of this technology is that the particles are intrinsically encoded by virtue of the difference in reflectivity of adjacent metal stripes. See FIG. 1.Any suitable encoded metal particles can be used to label metal surfaces in embodiments of the present invention. In one embodiment, the particles are segmented micro- or nanoscale particles such as those described in U.S. patent application Ser. No. 09/677,198, “Assemblies Of Differentiable Segmented Particles,” and U.S. patent application Ser. No. 09/677,203, “Methods of Manufacturing Colloidal Rod Particles as Nanobar Codes,” both filed Oct. 2, 2000, and both incorporated herein by reference. These particles are referred to as Nanobarcodes® particles.

Nanobarcodes particles are defined in part by their size and by the existence of at least 2 segments. The length of the particles can be from 10 nm to 50 μm. In some embodiments the particle is 500 nm to 30 μm in length. In the other embodiments, the length of the particles of this invention is 1 to 15 μm. The width, or diameter, of the particles of the invention is within the range of 5 nm to 50 μm. In some embodiments the width is 10 nm to 1 μm, and in other embodiments the width or cross-sectional dimension is 30 to 500 nm.

The Nanobarcodes particles are frequently referred to as being “rod” shaped. However, the cross-sectional shape of the particles, viewed along the long axis, can have any shape. The Nanobarcodes particles contain at least two segments, and as many as 50. In some embodiments, the particles have from 2 to 30 segments and most preferably from 3 to 20 segments. The particles may have from 2 to 10 different types of segments, preferably 2 to 5 different types of segments. A segment of the particle is defined by its being distinguishable from adjacent segments of the particle.

As discussed above, the Nanobarcodes particles are characterized by the presence of at least two segments. A segment represents a region of the particle that is distinguishable, by any means, from adjacent regions of the particle. In preferred embodiments, the segments are composed of different materials and segments are distinguishable by the change in composition along the length of the particle. In particularly preferred embodiments, the segments are composed of different metals. Segments of the particle bisect the length of the particle to form regions that have the same cross-section (generally) and width as the whole particle, while representing a portion of the length of the whole particle. In some embodiments, a segment is composed of different materials from its adjacent segments. However, not every segment needs to be distinguishable from all other segments of the particle. For example, a particle could be composed of 2 types of segments, e.g., gold and platinum, while having 10 or even 20 different segments, simply by alternating segments of gold and platinum. A particle of the present invention contains at least two segments, and as many as 50. The particles may have from 2 to 30 segments and or in other embodiments may have 3 to 20 segments. The particles may have from 2 to 10 different types of segments, preferably 2 to 5 different types of segments. An advantage of using Nanobarcodes particles as the encoded substrates is the highly multiplexed capabilities, e.g., 9 stripes of 3 metals=˜10,000 combinations. Software has been developed (NBSee™ software) that rapidly decodes the identity of the particles imaged with an extremely high level of accuracy. In some embodiments, the NBSee software is used to analyze the assay proposed here.

A segment of the particle is defined by its being distinguishable from adjacent segments of the particle. The ability to distinguish between segments includes distinguishing by any physical or chemical means of interrogation, including but not limited to electromagnetic, magnetic, optical, spectrometric, spectroscopic and mechanical. In certain embodiments of the invention, the method of interrogating between segments is optical (reflectivity).

Adjacent segments may even be of the same material, as long as they are distinguishable by some means. For example, different phases of the same elemental material, or enantiomers of organic polymer materials can make up adjacent segments. In addition, a rod comprised of a single material could be considered a Nanobarcode particle if segments could be distinguished from others, for example, by functionalization on the surface, or having varying diameters. Also particles comprising organic polymer materials could have segments defined by the inclusion of dyes that would change the relative optical properties of the segments. In certain embodiments of the invention, the particles are “functionalized” (e.g., have their surface coated with IgG antibody or oligonucleotide). Such functionalization may be attached on selected or all segments, on the body or one or both tips of the particle. The functionalization may actually coat segments or the entire particle. Such functionalization may include organic compounds, such as an antibody, an antibody fragment, or an oligonucleotide, inorganic compounds, and combinations thereof. Such functionalization may also be a detectable tag or comprise a species that will bind a detectable tag. Examples of functionalization are described herein. In some embodiments, the functional unit or functionalization of the particle comprises a detectable tag. A detectable tag is any species that can be used for detection, identification, enumeration, tracking, location, positional triangulation, and/or quantitation. Such measurements can be accomplished based on absorption, emission, generation and/or scattering of one or more photons; absorption, emission generation and/or scattering of one or more particles; mass; charge; faradoic or non-faradoic electrochemical properties; electron affinity; proton affinity; neutron affinity; or any other physical or chemical property, including but limited to solubility, polarizability, melting point, boiling point, triple point, dipole moment, magnetic moment, size, shape, acidity, basicity, isoelectric point, diffusion coefficient, or sedimentary coefficient. Such molecular tag could be detected or identified via one or any combination of such properties.

The composition of the particles is best defined by describing the compositions of the segments that make up the particles. A particle may contain segments with extremely different compositions. For example, a single particle could be comprised of one segment that is a metal, and a segment that is an organic polymer material.

The segments of the present invention may be comprised of any material. In preferred embodiments of the present invention, the segments comprise a metal (e.g., silver, gold, copper, nickel, palladium, platinum, cobalt, rhodium, iridium); any metal chalcognide; a metal oxide (e.g., cupric oxide, titanium dioxide); a metal sulfide; a metal selenide; a metal telluride; a metal alloy; a metal nitride; a metal phosphide; a metal antimonide; a semiconductor; a semi-metal. A segment may also be comprised of an organic mono- or bilayer such as a molecular film. For example, monolayers of organic molecules or self assembled, controlled layers of molecules can be associated with a variety of metal surfaces.

A segment may be comprised of any organic compound or material, or inorganic compound or material or organic polymeric materials, including the large body of mono and copolymers known to those skilled in the art. Biological polymers, such as peptides, oligonucleotides and polysaccharides may also be the major components of a segment. Segments may be comprised of particulate materials, e.g., metals, metal oxide or organic particulate materials; or composite materials, e.g., metal in polyacrylamide, dye in polymeric material, porous metals. The segments of the particles of the present invention may be comprised of polymeric materials, crystalline or non-crystalline materials, amorphous materials or glasses.

Segments may be defined by notches on the surface of the particle, or by the presence of dents, divits, holes, vesicles, bubbles, pores or tunnels that may or may not contact the surface of the particle. Segments may also be defined by a discernable change in the angle, shape, or density of such physical attributes or in the contour of the surface. In embodiments of the invention where the particle is coated, for example with a polymer or glass, the segment may consist of a void between other materials.

The length of each segment may be from 10 nm to 50 μm. In some embodiments the length of each segment is 50 nm to 20 μm. Typically, the length. is defined as the axis that runs generally perpendicular to lines defining the segment transitions, while the width is the dimension of the particle that runs parallel to the line defining the segment transitions. The interface between segments, in certain embodiments, need not be perpendicular to the length of the particle or a smooth line of transition. In addition, in certain embodiments the composition of one segment may be blended into the composition of the adjacent segment. For example, between segments of gold and platinum, there may be a 5 nm to 5 μm region that is comprised of both gold and platinum. This type of transition is acceptable so long as the segments are distinguishable. For any given particle the segments may be of any length relative to the length of the segments of the rest of the particle.

As described above, the particles can have any cross-sectional shape. In preferred embodiments, the particles are generally straight along the lengthwise axis. However, in certain embodiments the particles may be curved or helical. The ends of the particles may be flat, convex or concave. In addition, the ends may be spiked or pencil tipped. Sharp-tipped embodiments of the invention may be preferred when the particles are used in Raman spectroscopy applications or others in which energy field effects are important. The ends of any given particle may be the same or different. Similarly, the contour of the particle may be advantageously selected to contribute to the sensitivity or specificity of the assays (e.g., an undulating contour will be expected to enhance “quenching” of fluorophores located in the troughs).

In the present invention, some embodiments of these particles are segmented cylindrical or rod-shaped particles formed from segments of different metals (e.g., gold and silver), which have different light reflectivities at given wavelengths. As a result, reflectance images of the particles appear striped, and the particles are considered to be encoded with a striping pattern. By varying the number of materials, stripes, and stripe thicknesses, a large number of striping patterns may be formed. Combining particles into groups of differently-coded particles increases the number of codes dramatically. Particles can be manufactured by, e.g., sequentially electroplating segments of different metals into templates and releasing the resulting particles from the templates.

When the particles are made by electrochemical deposition the length of the segments (as well as their density and porosity) can be adjusted by controlling the amount of current passed in each electroplating step; as a result, the rod resembles a “bar code” on the nanometer scale, with each segment length (and identity) programmable in advance. Other forms of electrochemical deposition can also yield the same results. For example, deposition can be accomplished via electroless processes and by controlling the area of the electrode, the heterogeneous rate constant, the concentration of the plating material, and the potential. The same result could be achieved using another method of manufacture in which the length or other attribute of the segments can be controlled. While the diameter of the rods and the segment lengths are typically of nanometer dimensions, the overall length is such that in preferred embodiments it can be visualized directly in an optical microscope, exploiting the differential reflectivity of the metal components. The synthesis and characterization of multiple segmented particles is described in Martin et al., Adv. Materials 11: 1021-25 (1999). The article is incorporated herein by reference in its entirety.

Application and readout of particles may take place manually (with an optical microscope, exploiting the differential reflectivity of the particle components, including metal components). Alternatively, both application and readout can be performed automatically. In particular, automated image processing methods can be employed to determine the code of each particle and verify the identity of the labeled object. In the case of the segmented particles described above, suitable methods, including, but not limited to absorbance, fluorescence, Raman, hyperRaman, Rayleigh scattering, hyperRayleigh scattering, CARS, sum frequency generation, degenerate four wave mixing, forward light scattering, back scattering, or angular light scattering), scanning probe techniques (near field scanning optical microscopy, AFM, STM, chemical force or lateral force microscopy, and other variations), electron beam techniques (TEM, SEM, FE-SEM), electrical, mechanical, and magnetic detection mechanisms (including SQUID), are described in U.S. patent application Ser. No. 09/676,890, “Methods of Imaging Colloidal Rod Particles as Nanobar Codes,” filed Oct. 2, 2000, incorporated herein by reference. It may be necessary to tailor software parameters for imaging a particular metal surface to which the particles are attached.

Micro- or nanoscale particles lend themselves to a number of methods for brand security, e.g., blended in a variety of label-specific host mediums such as inks and varnishes and affixed to items. Encoded Nanobarcodes particles, for instance, can be used in serialized tags for track-and-trace applications. The unique characteristics of Nanobarcodes particles (e.g., striping pattern, length, diameter) allows differentiable groups of particles to be created, each group constituting a “type” or “flavor” of particle. Particles of a specific flavor then can be used, alone or in combination with particles of one or more other flavors, to uniquely tag an item. Such methods rely on matching a specific tag to a specific item. In a typical application, the Nanobarcodes particles are synthesized and pre-sorted into groups according to type or flavor before being affixed to an item. The item can be optically examined at a later date to determine the flavor of the affixed particle. In many cases, this is sufficient. However, in some cases, depending on the complexity of the code and the number of different tags required, the method may involve rather sophisticated particle handling technology. In many cases, ink and varnish presses do not have the equipment necessary to accommodate microvolume sorting and handling.

The Nanobarcode particles are made in one embodiment by electrochemical deposition in an alumina or polycarbonate template, followed by template dissolution, and typically, they are prepared by alternating electrochemical reduction of metal ions, though they may easily be prepared by other means, both with or without a template material. In the case of the segmented particles described above, suitable methods are described in U.S. patent application Ser. No. 09/677,203, “Method of Manufacture of Colloidal Rod Particles as Nanobar Codes,” filed Oct. 2, 2000, incorporated herein by reference. The Nanobarcodes particles are manufactured in a semi-automated, highly scalable process by electroplating inert metals—such as gold (Au), nickel, platinum (Pt), or silver (Ag)—into templates that define the particle diameter, and then releasing the resulting striped nano-rods from the templates. Just as a conventional barcode is read by measuring the differential contrast between adjacent black and white lines using an optical scanner, individual Nanobarcodes particles are read by measuring the differential reflectivity between adjacent metal stripes within a single particle using a conventional optical microscope.

Genomic assays have been performed with Nanobarcodes particles where the particles serve as encoded substrates. See Penn, S. G., Hel, L., and Natan, M. J., “Nanoparticles for bioanalysis.” Curr Opin Chem Biol 2003, 7, 609-615. This approach achieves very high levels of multiplexing, but typically requires the biomolecule in question to be labeled or an additional label to be added to the system. The present invention takes advantage of the fact that the metallic Nanobarcodes particles will quench fluorescence emission. By attaching the probe oligonucleotide to the Nanobarcodes particles, fluorescence quenching may be used as the means for detection and the advantages of sensitive fluorescence detection are achieved without analyte labeling. Because the Nanobarcodes particles are encoded, the assay may be multiplexed even using a single fluorophore. In these embodiments, the molecular beacon pair may be excited by a quantum of electromagnetic radiation at a wavelength at which a fluorochrome member of the pair is excited; however, fluorescence from the fluorochrome that would be expected in the absence of the metallic Nanobarcodes particles is quenched at least in part. When the flourochrome and the metallic Nanobarcodes particles are in close proximity, the quenching due to the metallic Nanobarcodes particle the prevents detection of a fluorescent signal. When the flourochrome and Nanobarcodes particle is separated, however, the fluorescent signal becomes detectable.

Preferably, the methods used to produce the particles derivatized with oligonucleotides are well-controlled. Protocols for the attachment of both biotin and amine derivatized oligonucleotides to Nanobarcodes particles (using carbodiimide attachment chemistry previously have been developed and characterized). However, the quenching effect of the metallic particle upon fluorescent oligonucleotide, as described herein, has not been observed with either of these linkage chemistries. This is most likely because these large moieties effectively block the surface of the Nanobarcodes particle.

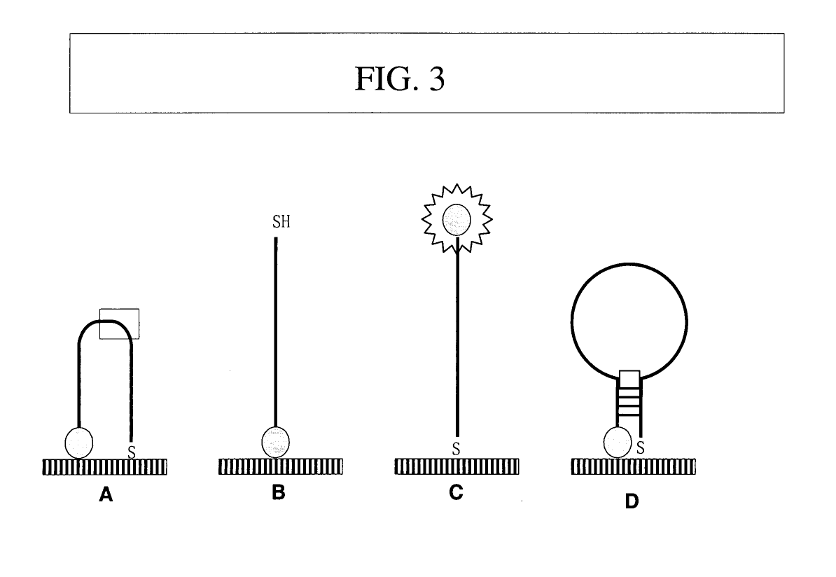

A number of different configurations could possibly occur when attempting to couple fluorescent oligonucleotides to a particle. FIG. 3A shows the desired orientation in which the probe oligonucleotide has successfully coupled to the particle surface through a thiol linkage. The fluorophore is in close proximity to the particle surface and is quenched. FIG. 3B shows an unsuccessful attachment of the oligonucleotide where the fluorophore has adsorbed onto the particle surface. The oligonucleotide has not coupled to the particle surface through a thiol linkage. However, because the fluorphore is in close proximity to the particle surface, this orientation will also result in a quenching of the fluorophore. FIG. 3C shows the probe oligonucleotide has successfully coupled to the particle surface through a thiol linkage, but the fluorophore is not in close proximity to the particle surface. This might occur for a number of reasons, including electrostatic repulsion of the dye, or steric hindrance resulting from oligonucleotides being packed too closely together. There is insufficient interaction between the surface and the dye and the fluorescent dye will not be quenched.

The configuration shown in FIG. 3C largely can be eliminated by using an internal “hairpin” sequence (molecular beacon) that will constrain the possible positions of the fluorophore relative to the particle surface. A “hairpin” is the structure formed by a polynucleic acid by base-pairing between neighboring complementary sequences of a single strand of either DNA or RNA. The “hairpin loop is area where single-stranded DNA or RNA has folded and nucleotides from the 3′ and 5′ segments have base paired, so that the resulting structure appears as the name describes. A hairpin structure is shown in FIG. 3D.

Distinguishing the successful configuration shown in FIG. 3A with the unsuccessful configuration shown in FIG. 3B, or the failure to couple, presents a challenge for quality control. All three scenerios will appear “dark” to interrogation by fluorescence-based microscopy. However, a number of approaches may be used to address this problem.

The literature contains ample disclosure concerning the use of fluorescent dyes with metal surfaces. Using the teachings of the present invention, it is within the skill of one in the art to select and optimize the organic fluorescent dye to use in the present invention.

Due to the size of the Nanobarcodes particles (typically microns by several hundred nanometers) they behave more like Au films than Au colloids.

Nanobarcodes particles may be composed of a number of different metals. There may be differences in non-radiative fluorescence quenching of the different metals (e.g., Au vs. Ag vs. Pt surfaces). Theory suggests that most metals will quench visible fluorescence when the fluorophore is in direct contact with a metal. Because Au and Ag have different surface plasmon bands, the degree of quenching for any fluorophore may vary and a stripe pattern may appear. One of skill in the art would understand how to optimize such a system based on the teachings of the present invention, and the published literature. For example, rhodamine fluorescence is quenched by Au and Ag.

The fluorescence quenching may be quantified by direct monitoring of the fluorophore behavior over time. It is known that the fluorescence lifetime of a fluorophore is modified when it is quenched. Thus, fluorescence lifetime data of a given system may be taken into account to understand the changes undergone by the fluorophores upon binding. High-resolution fluorescent lifetime images may be taken to understand the distribution of lifetime changes across the particles. Lifetime images may be taken of the listed fluorophores in order to understand which are the most sensitive to metal quenching by Au, Ag and Pt.

It is also possible to determine optimum quenching by vary the distance of the fluorophores from the metal surface. This distance may be controlled, for example, by using alternating layers of biotin-BSA and avidin. Alternatively, the distance may be controlled by using dye labeled alkanethiols of varying lengths. Such dye labeled alkanethiols may be synthesized or purchased from a commercial source.

From the measurements obtained from the optimization strategies outlined above, the theoretical limit of detection of the assay of the invention may be determined. When using Nanobarcodes particles as the particle, it is possible to use dyes with fluorescent emission maxima away from the wavelength where reflectance of the particle is measured (400 nm). Dyes with fluorescent emission maxima close to the wavelength where reflectance is measured could affect the accuracy of the software to quantitate fluorescence. Sometimes, if fluorescence is close to where reflectance is measured (400 nm), the striping pattern in the fluorescence (a “leak through” of the reflectance signal into the fluorescence channel) could interfere with quantitation.

Due to the repulsion between the phosphate groups on the DNA molecules, DNA molecules likely do not form self-assembled monolayers by simple adsorption. See Huang, E., Satjapipat, M., Han, S., and Zhou, F., “Surface Structure and Coverage of an Oligonucleotide Probe Tethered onto a Gold Substrate and Its Hybridization Efficiency for a Polynucleotide Target” Langmuir 2001, 17, 1215-1224. The density of adsorption is reported to be a function of oligonucleotide length and absorption procedure. There is abundant literature with respect to methods of attachment of thiol oligonucleotides to metallic surfaces. Au films have been immersed in mixtures of thiolated DNA and mercaptopropanol. DNA has been absorbed to surfaces and subsequently blocked with mercaptohexanol.

Oligonucleotides may be absorbed to Au nanoparticles in solution for 24 hours, followed by a titration with phosphate buffer and sodium chloride for an additional 40 hours.

A number of aspects of the coupling protocols may be adjusted to optimize the surface coverage and composition in a particular instance. For example, the length of the oligonucleotide may be varied, the time and temperature of the adsorption may be varied, and “spacer” groups between the particle and the probe oligonucleotide may be used (e.g., alkanethiols or polynucleotides). The density of the packing of the probe oligonucleotides on the particle will have a significant impact on the subsequent quenching ability and hybridization efficiency. If the packing is too close, the probe oligonucleotides will be sterically restrained in such a way that they will not be quenched (e.g., they will not be able to form hairpins, or the fluorophore will not have ready access to the metal surface). Furthermore, such close packing may interfere with hybridization between the target olignucleotides and the sterically restrained probe oligonucleotides and thus lower hybridization efficiency. It is important to maximize the hybridization efficiency because this will maximize the dynamic range and detection limit of the assay. Thus, close packing should be avoided.

A number of methods may be used to monitor progress of the coupling of the oligos to the particle. For example, the oligonucleotides may be displaced from the surface of the particle using mercaptoethanol or other thiol containing molecules via an exchange reaction. Detailed protocols for displacement of thiol-derivatized oligonucleotides from Au colloids and films are available to one of ordinary skill in the art. These methods may be optimized for Nanobarcodes particles by carrying out time and temperature course evaluations for a series of mercaptoethanol concentrations to determine the end point of the reaction.

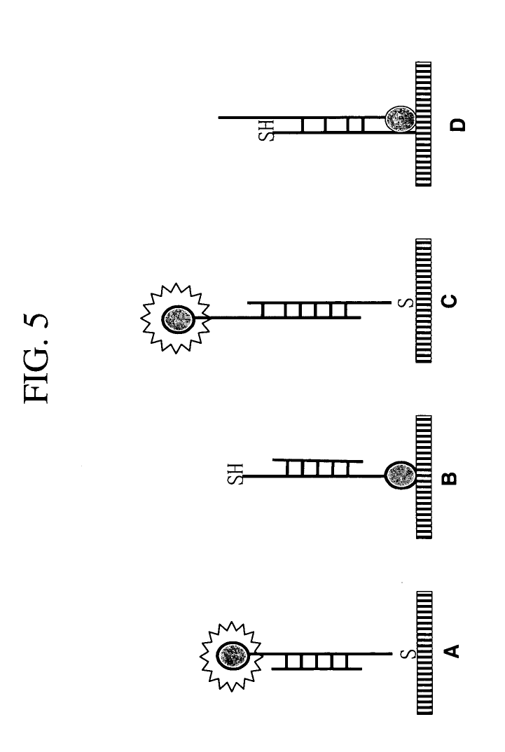

An alternative approach for verifying the successful attachment of the oligonucleotide to the surface uses a of “pre-hybridized” oligonucleotides, i.e., probe oligonucleotides that already have been hybridized to a complementary sequence prior to being attached to the particle surface. The double-stranded oligonucleotides have more rigidity and so in a successfully attached conformation, the fluorophore will not interact with the particle surface and thus will not be quenched. Accordingly, a successful linkage to the surface will result in fluorescence. See FIG. 5A. However, in a miscoupling will result in quenching. See FIG. 5B.

Another alternative approach for verifying successful attachment of the oligonucleotide to the surface is (a) to couple unlabelled thiol-linked probe oligonucleotides to the surface of the particle, and then (b) to hybridize the probe oligonucleotides with complementary oligonucleotides that have been fluorescently labeled. A successful coupling followed by successful hybridization will result in fluorescence. See FIG. 5C. However, a miscoupling followed by hybridization would result in the fluorescence being quenched. See FIG. 5D.

Significantly, none of the foregoing methods is able to determine the result from a correctly oriented fluorophore that is not interacting with the surface, as was depicted in FIG. 3C. Accordingly, the hairpin loops are utilized in some embodiments because they eliminate one way that a false positive can occur (albeit an unlikely one) Therefore, to minimize this problem, in some embodiments, the oligonucleotides are constructed so that they have hairpin loops. The hairpin loops force the interaction between the fluorophore and the particle surface.

As described above, the present invention provides an assay in which fluorescence intensity increases upon hybridization and fluorescence intensity remains unchanged in a negative control experiment. Parameters of an individual assay may be optimized by adjusting the buffer conditions, hybridization times, hybridization temperatures, oligonucleotide sequence requirements, thiol-Au bond stability, and number and character of stringency washes. As used herein, stringent hybridization conditions refer to standard hybridization conditions under which nucleic acid molecules, including oligonucleotides, are used to identify molecules having similar nucleic acid sequences. Such standard conditions are disclosed, for example, in Sambrook et al., Molecular Cloning: A Laboratory Manual, Cold Spring Harbor Labs Press (1989). Sambrook et al., is incorporated by reference herein in its entirety. Stringent hybridization conditions typically permit isolation of nucleic acid molecules having at least about 70% nucleic acid sequence identity with the nucleic acid molecule being used to probe in the hybridization reaction. Formulae to calculate the appropriate hybridization and wash conditions to achieve hybridization permitting 30% or less mismatch of nucleotides are disclosed, for example, in Meinkoth, J. et al., Anal. Biochem. 138:267-284 (1984); Meinkoth, J. et al., ibid., is incorporated by reference herein in its entirety. In some embodiments, hybridization conditions will permit hybridization of nucleic acid molecules having at least about 80% nucleic acid sequence identity with the nucleic acid molecule being used to probe. In other embodiments, hybridization conditions will permit isolation of nucleic acid molecules having at least about 90% nucleic acid sequence identity with the nucleic acid molecule being used to probe. In other embodiments, hybridization conditions will permit isolation of nucleic acid molecules having at least about 95% nucleic acid sequence identity with the nucleic acid molecule being used to probe.

One of skill in the art will also be informed by the body of work on fundamental studies on the behavior of nanoparticle-biomolecule and surface-biomolecule interactions. For example, a systematic study of hybridization efficiencies of DNA attached to 12 nm Au nanoparticles has been carried out to characterize the effect of space length, concentration, complement length and oligonucleotide length. In addition, a very thorough model has been provided of the behavior of DNA hybridization in the presence of Au nanoparticles (ranging in size from 13nm to 50 nm) that explains the sharp hybridization transition temperature observed (which is sharper than observed in an untagged DNA duplex).

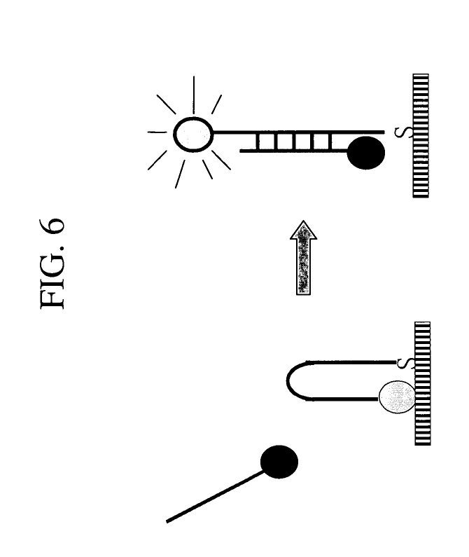

A number of methods may be used to verify successful hybridization. For example, hybridization may be carried out by using a labeled target nucleic acid, as shown in FIG. 6. The particle-bound probe oligonucleotide is contacted with the labeled target nucleic acid and the labeled nucleotide hybridizes with the probe oligonucleotide. Following hybridization and stringency washes, the fluorescence signal of Nanobarcodes particles in the reaction is determined. By increasing the temperature and lowering the salt concentration, the double-stranded oligonucleotide may be “melted” to release the labeled target nucleic acid. By centrifuging the reaction and quantitating the fluorescence of the eluent, the amount of oligonucleotides hybridized can be determined. Following this, the oligonucleotides bound on the surface can be displaced with an alkanethiol and the eluent collected and the fluorescence measured. This method will allow the determination of both surface coverage and hybridization efficiency, from the same particles.

The Au-thiol bond is stable under high salt conditions (0.5 M NaCl). Furthermore, the biologically relevant conditions under which the Au-thiol, Ag-thiol and Pt-thiol bonds are stable may be further characterized by determining the effect of varying the temperature from about 25° C. to about 70° C., the effect of varying salt concentration from about 0 to about 1 M, and the effect of the inclusion of about 0 to about 10% SDS detergent and about 0 to about 50% formamide. See Example ______.

In many hybridization assays that occur on a surface, a “spacer” is needed to move the interrogated sequence away from the surface so that the hybridization can occur sterically unhindered. This effect has been reported on planar surfaces, including microarrays, as well as on colloidal Au. Significantly, the present invention comprises particles that are considerably larger than Au colloids and therefore act more like planar surfaces. The spacer groups may be varied from about 0 to about 20 bases on the nucleic acid sequence, if nucleic acid spacers, and a spacer of the same length, if a hydrocarbon spacer is used. When a spacer is desired, C₆(CH₂)ₓ may be used. It is important that the length of the spacer (if any) and the oligonucleotide probe are sufficient to allow the fluorophore to come within the required distance for quenching. Longer spacers (if any) and oligonucleotide probes are within the scope of the invention. However the longer the oligonucleotide probe, the more expensive it is to synthesize, and the more likely it is that the probe will contain sequences that are self-complementary and/or that compete with the target nucleic acid.

The present invention includes both hairpin configurations and non-hairpin configurations. The use of hairpin sequences, of course, requires internal complementary sequences to form the hairpin, and thus puts some constraints on the overall sequence of the prove oligonucleotide. See Dubertret et al., 2001. Non-hairpin configurations result in quenching because the oligonucleotide is flexible and the negatively charged fluorophore will tend to reside in close proximity to the positively charged metal surface. See Mawell et al., 2002.

The sequence lengths of the probe oligonucleotides may be any length that permits acceptable robustness and reproducibility. The methods, such as those described above, may be used to determine both hybridization efficiency and the effect of length on surface coverage. However, in particular, the sequences may be of between about 8 and about 100 bases in length. When the assay conditions are optimized multiple experiments may be performed in which a dilution series of a PCR product is assayed, to investigate the linearity, dynamic range and sensitivity of a single component assay on the Zeiss microscope system.

### EXAMPLES

The following Experiments have been performed to illustrate the invention.

Example 1

Preparation of Nanobarcodes Particles Conjugated with Thiol Oligo.

Previously prepared Nanobarcodes particles are stored in double deionized H₂O after QC with concentration 1×10⁹ NBC/ml. Wash 100 μL NBC twice with 10 mM Phosphate Buffered Saline, pH=7.4 (PBS) (Sigma cat#P-3813) and re-suspend with 100 μL PBS. Add 500 μL of 5 μM probe thio-labeled DNA oligo (in double deionized H₂O) and store overnight at room temperature to allow probe to self-assemble on NBC. Add 600 μL of 0.3M NaCl/10 mM PBS and store for two hours room temperature. Wash with 0.3M NaC/10 mM PBS. Add 100 μL PBS to re-suspend the NBC and store at 4° C. which is ready for hybridization.

### Example 2

Hybridization and Reading of Result. Combine 90 μl hybridization buffer (HS114, Molecular Research Center, Inc.), 10 μL of 10 μM target oligo, 3 μL NBC-probe. Shake at 42° C. for 1 hr. Wash once with 1×SSC and once with 0.1×SSC one time. Add 30 μL 5 mM PBS and image with microscopy.

### Example 3

Non-hairpin loop. A 32-mer oligonucleotide (M1) labeled with carboxytetramethylrhodamine (TAMRA) was conjugated to a Nanobarcodes particle (NBC). The resulting conjugated particle (NBC-M1) was incubated with a complementary sequence (M1C), a non-complementary sequence (M2C) and a water control. Upon hybridization with its complementary sequence (M1C), the fluorescence signal from the TAMRA label was over 200 MFI, compared to less than 50 MFI in the water and non-complementary controls. FIG. 4A. A similar experiment was carried out in which a different oligonucleotide (M2) labeled with TAMRA was conjugated to a NBC. The resulting conjugated particle (NBC-M2) was incubated with a complementary sequence (M2C), a non-complementary sequence (M1C), and a water control. Upon hybridization with its complementary sequence, the fluorescence signal from the TAMRA label was over 200 MFI compared to less than 50 MFI in the water and non-complementary controls. FIG. 4B. These results indicate that fluorescence quenching is occurring unless the conjugated oligonucleotide is hybridized to its complementary sequence.

### Example 4

Multiplexed Assays

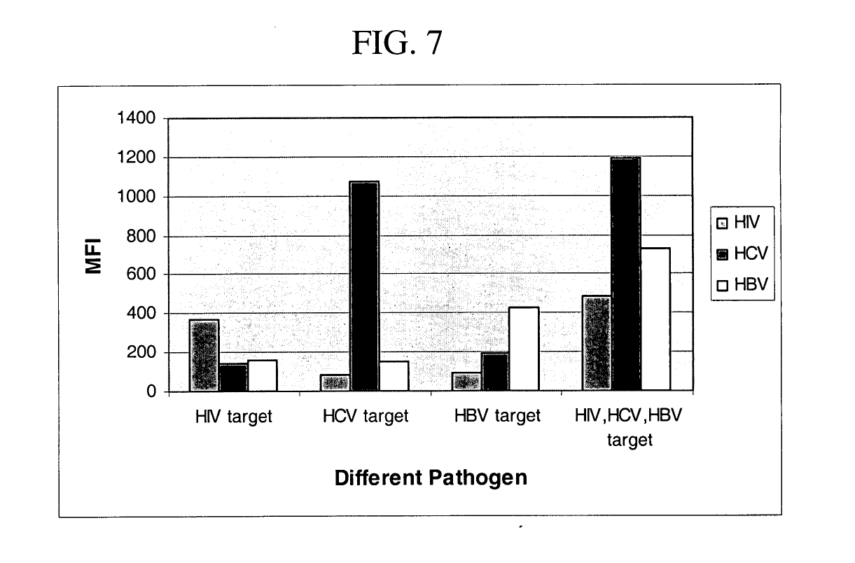

Nanobarcodes particles were conjugated with either TAMRA-labeled HIV probe oligonucleotide or TAMRA-labeled HCV oligonucleotide. As shown in FIG. 7, Nanobarcodes particles conjugated to TAMRA labeled HCV oligonucleotide probes were found to exhibit far greater fluorescence when contacted with complementary HCV target oligonucleotide compared to (noncomplementary) HIV target oligonucleotide or water. FIG. 7 (panel B). Similarly, Nanobarcodes particles conjugated to TAMRA labeled HIV oligonucleotide probes were found to exhibit greater fluoresce when contacted with complementary HIV target oligonucleotide compared to (noncomplementary) HBC oligonucleotide or water. FIG. 7 (panel A).

In another analysis, the probe sequences were linked to different types of encoded particles, as follows: The HIV probe sequence (HIV mb2) was conjugated to Nanobarcodes particles with code (00001); the HCV probe sequence (HCV mb2)was conjugated to Nanobarcodes particles with code (00010); the HBV probe sequence (HBV mb2) was conjugated to Nanobarcodes particles with code (01100).

The conjugated Nanobarcodes particles were then exposed to the target sequences shown in Table 1 (i.e., HIV mb1c, HCV mb1c, HBV mb1c). As shown in FIG. 8, in each case, the greatest fluorescence was observed for the Nanobarcodes particles that were conjugated to the TAMRA-labeled oligonucleotides that were complementary to the target oligonucleotide at issue.

TABLE 1

| | SEQUENCE | | |
|---|---|---|---|
| CLASSIFICATION | NAME | SEQUENCE | Tₘ |
| Probe | HIV mb2 | 5′TAMRA (CH₂)₇ gcgag GAGACCATCAA | 87 |
| | | TGAGGAAGCTGCA ctcgc (CH₂)₆ thiol-3′ | |
| | | (SEQ ID NO:1) | |
| Probe | HCV mb2 | 5′TAMRA (CH₂)₇ gcgag | 89 |
| | | CATAGTGGTCTGCGGAACCGGTGA ctcgc | |
| | | (CH₂)₆ thiol-3′ | |
| | | (SEQ ID NO:2) | |
| Probe | HBV mb2 | 5′TAMRA (CH₂)₇ gcgag | 83 |
| | | AATCTCGGGAATCTCAATGTTAGT ctcgc | |
| | | (CH₂)₆ thiol-3′ | |
| | | (SEQ ID NO:3) | |
| Target | HIV mb1c | TGCAGCTTCCTCATTGATGGTCTC | 77 |
| | | (SEQ ID NO:4) | |
| Target | HCV mb1c | TCACCGGTTCCGCAGACCACTATG | 80 |
| | | (SEQ ID NO:5) | |
| Target | HBV mb1c | ACTAACATTGAGATTCCCGAGATT | 72 |
| | | (SEQ ID NO:6) | |

The oligonucleotides were obtained from Biosource International, Camarillo, Calif. The sequences were selected from Perrin A., et al. Analytical Biochemistry, 2003, 322, 148-155. The HIV sequences codes for the gag glycoprotein gene; the HBV sequence codes for polymerase.

1. An encoded metal nanoparticle comprising an oligonucleotide, said oligonucleotide comprising a fluorophore.

2. The encoded metal nanoparticle of claim 1 wherein the encoded metal nanoparticle comprises at least one metal selected from the group consisting of gold, silver, and platinum.

3. The encoded metal nanoparticle of claim 1, wherein the oligonucleotide is associated with the encoded metal nanoparticle via a thiol linkage.

4. The encoded metal nanoparticle of claim 1, further comprising a spacer group wherein the spacer group moves the oligonucleotide away from the surface of the encoded metal nanoparticle.

5. The encoded metal nanoparticle of claim 1, wherein the oligonucleotide is a hairpin oligonucleotide.

6. An assembly of encoded metal particles comprising a plurality of types of particles, wherein each particle is from 10 nm to 50 μm in length and is comprised of a plurality of segments, and wherein at least one of said types is differentiable from another of said types based on the sequence of said segments, and wherein each particle comprises an oligonucleotide, said oligonucleotide comprising a fluorophore.

7. The assembly of encoded metal particles of claim 6 wherein at least one of said types is differentiable from another of said types by optical means, electrical means, physical means, chemical means or magnetic means.

8. The assembly of encoded metal particles of claim 7 wherein at least one of said types is differentiable from another of said types by optical means.

9. The assembly of encoded metal particles of claim 8 wherein at least one of said types is differentiable from another of said types by differential reflectivity.

10. The assembly of encoded metal particles of claim 6 wherein each said particle comprises 2 to 50 segments, and wherein the length of each particle is from 1 to 15 μm, the width of each particle is from 30 nm to 2 μm, and the segment lengths are from 50 nm to 10 μm.

11. The assembly of encoded metal particles of claim 6 comprising at least one type that comprises a different oligonucleotide from another type.

12. A method for preparing a nanoparticle comprising:
a) providing an encoded metal nanoparticle
b) conjugating an oligonucleotide to the encoded metal nanoparticle.

13. The method of claim 12, wherein said oligonucleotide comprises a free thiol group, and wherein said conjugating comprises the formation of a metal-sulfur bond.

14. The method of claim 12, wherein the oligonucleotide comprises a fluorphore.

15. The method of claim 12, further comprising assessing the conjugation, wherein said assessing comprises:
a) providing a oligonucleotide complementary to the oligonucleotide conjugated to the encoded metal nanoparticle;
b) illuminating the encoded metal nanoparticle with light capable of stimulating fluorescence from the fluorophore;
c) detecting fluorescence transmitted from the encoded metal nanoparticle, wherein an increase in fluorescence indicates conjugation.

16. The method of claim 12, wherein the oligonucleotide is an oligonucleotide selected from the group consisting of SEQ ID NO:1, SEQ ID NO:2, SEQ ID NO:3, SEQ ID NO:4, SEQ ID NO:5, SEQ ID NO:6.

17. A method for detecting a target nucleic acid, comprising:
a) providing an encoded metal nanoparticle comprising an oligonucleotide, said oligonucleotide comprising a fluorophore;
b) contacting the target nucleic acid with the encoded metal nanoparticle comprising an oligonucleotide under conditions permitting hybridization; and
c) detecting hybridization.

18. The method of claim 17, wherein said hybridization is detected by an increase in fluorescence.

19. The method of claim 17, wherein said detecting hybridization is performed under stringent hybridization conditions.

20. A method of comparing a target nucleic acid with a reference nucleic acid comprising:
a) contacting the target nucleic acid and the reference nucleic acid with an encoded metal nanoparticle comprising an oligonucleotide, said oligonucleotide comprising a fluorophore, under conditions permitting hybridization,
b) determining a level of hybridization with the target nucleic acid and a second level hybridization with the reference nucleic acid, and
c) comparing the first and second levels of hybridization to determine similarities or differences between the target nucleic acid and the reference nucleic acid.

21. The method of claim 20, wherein said hybridization is detected by an increase in fluorescence.

22. The method of claim 20, wherein said detecting hybridization is performed under stringent hybridization conditions.

23. A method of detecting a polynucleotide, the method comprising:
a) contacting a sample polynucleotide with an encoded metal nanoparticle comprising an oligonucleotide, said oligonucleotide comprising a fluorophore;
b) illuminating the encoded metal nanoparticle with light capable of stimulating fluorescence from the fluorophore;
c) detecting fluorescence transmitted from the encoded metal nanoparticle;
d) deriving information relating to the extent of hybridization between the sample polynucleotide and the encoded metal nanoparticle based on the amount of fluorescence emitted from the hybrid.

24. The method of claim 23, wherein said hybridization is detected by an increase in fluorescence.

25. The method of claim 23, wherein said detecting hybridization is performed under stringent hybridization conditions.

26. A method for detecting a plurality of target nucleic acids, comprising:
a) providing an assembly of encoded metal particles comprising a plurality of types of particles, wherein each particle is from 10 nm to 50 μm in length and is comprised of a plurality of segments, and wherein at least one of said types is differentiable from another of said types based on the sequence of said segments, and wherein each particle comprises an oligonucleotide, said oligonucleotide comprising a fluorophore;
b) contacting plurality of target nucleic acid with the assembly of encoded metal particles under conditions permitting hybridization;
c) detecting hybridization; and
d) identifying the type of encoded metal particle which exhibits hybridization.

27. A method of detecting a plurality of polynucleotides, the method comprising:
a) contacting a sample polynucleotide with an assembly of encoded metal particles comprising a plurality of types of particles, wherein each particle is from 10 nm to 50 μm in length and is comprised of a plurality of segments, and wherein at least one of said types is differentiable from another of said types based on the sequence of said segments, and wherein each particle comprises an oligonucleotide, said oligonucleotide comprising a fluorophore;
b) illuminating assembly of encoded metal particles with light capable of stimulating fluorescence from the fluorophore;
c) detecting fluorescence transmitted from the encoded metal nanoparticles;
d) deriving information relating to the extent of hybridization between the sample polynucleotide and the encoded metal nanoparticle based on the amount of fluorescence emitted from the hybrid; and
e) identifying the type of encoded metal particle which exhibits hybridization.

28. A kit for use in detecting the presence in a sample of a nucleic acid sequence of interest, the kit comprising an encoded metal nanoparticle comprising an oligonucleotide, said oligonucleotide comprising a fluorophore, appropriate packaging means, and one or more containers for holding one or more components of the kit.

29. A kit according to claim 28, further comprising instructions for use in performing the method of claim 23.

30. A kit according to claim 28, further comprising one or more of the following: a DNA polymerase; an RNA polymerase; ribo- or deoxyribo-nucleotide triphosphates (labelled or unlabelled); labelling reagents; detection reagents; buffers.

31. A kit comprising the assembly of claim 6 and one or more of: packaging materials, instructions for using the assembly, one or more containers for holding one or more components of the assembly.


SEQUENCE LISTING (6 sequences)

SEQ ID NO: 1 - DNA, 34 bp, artificial
  probe
gcgaggagaccatcaatgaggaagctgcactcgc

SEQ ID NO: 2 - DNA, 34 bp, artificial
  probe
gcgagcatagtggtctgcggaaccggtgactcgc

SEQ ID NO: 3 - DNA, 34 bp, artificial
  probe
gcgagaatctcgggaatctcaatgttagtctcgc

SEQ ID NO: 4 - DNA, 24 bp, artificial
  target
tgcagcttcctcattgatggtctc

SEQ ID NO: 5 - DNA, 24 bp, artificial
  target
tcaccggttccgcagaccactatg

SEQ ID NO: 6 - DNA, 24 bp, artificial
  target
actaacattgagattcccgagatt